(19) United States
(12) Patent Application Publication
(10) Pub. No.: US 2016/0120572 A1
(43) Pub. Date: May 5, 2016

(54) Static pointing device

(71) Applicant: Choon Kee Lee, Denver, CO (US)

(72) Inventor: Choon Kee Lee, Denver, CO (US)

(21) Appl. No.: 14/527,736

(22) Filed: Oct. 29, 2014

(51) Int. Cl. A61B 17/34; A61B 8/00; A61B 8/08

(52) U.S. Cl. CPC A61B 17/3403; A61B 8/0841; A61B 8/4444; A61B 8/4281; A61B 2017/3413

(57) ABSTRACT

The present invention presents a static pointing device and methods to guide insertion of an invasive tubular device to a tissue object of a living body. The static pointing device is coupled with an ultrasound positioning apparatus, located in front of an ultrasound transducer and produces a linear shadow line in a visualized ultrasonographic field. An ultrasonographic view of a tissue object can be marked by the linear shadow line crossing said tissue object. An invasive tubular device held by the ultrasound positioning apparatus can be directed to the tissue object by calculated angulation of said invasive tubular device to have a longitudinal axis of said invasive tubular device cross the linear shadow line in the tissue object.

### CROSS REFERENCE TO RELATED APPLICATIONS

Attached please refer to the Information Disclosure Statement for the cross reference to related applications.

### STATEMENT REGARDING FEDERALLY SPONSORED RESEARCH OR DEVELOPMENT

The present invention is not a federally sponsored research or development.

### TECHNICAL FIELD

The present invention relates generally to the field of positioning guidance of insertion of invasive devices in a living body for medical purposes. More specifically, the present invention provides a static pointing device and methods to assist introduction of tubular devices into a tissue using ultrasound.

### BACKGROUND OF THE INVENTION

An invasive tubular device can be assisted for its insertion into a tissue object by an ultrasound guidance which is provided with a set of calculated numerical information of an insertion angle and a length of the invasive tubular device to reach the tissue object. Both the insertion angle and length of the invasive tubular device to get to the object can be calculated by a trigonometric measurement with a measured vertical depth from a point of a contact portion of an ultrasound transducer placed on a skin to an ultrasonographically visualized tissue object and a horizontal distance from the point of the contact portion of the transducer to a pivoting center of the invasive tubular device. Adjustment of an insertion angle of an invasive tubular device toward the tissue object can be coordinated with linear alignment between a point of the transducer head and the tissue object in an ultrasonographic field, which is accomplished by a position alignment assembly with a movably adjustable galvanometer-type electromagnetic pointing device. The galvanometer-type electromagnetic pointing device is located in between the ultrasound transducer head and the tissue object and comprises a movable linear pointer which is configured to block a portion of ultrasound waves emanating from the ultrasound transducer toward the tissue object, thereby producing a linear shadow line which can be readily recognizable in a visualized ultrasonographic field.

Although a linear movable pointer of an electromagnetic pointing device moves to a point of a skin overlying a tissue object to produce a linear shadow line from an ultrasound transducer to a tissue object whether the tissue object is off-center or on-center with a skin-contact portion of the ultrasound transducer, the device requires a source of electricity to drive the linear pointer with an electromagnetic force. In addition to an issue of a weight of the electromagnetic pointing device and the need of a power source, an electromagnetic field from said electromagnetic pointing device could potentially interfere with adjacent devices, which may adversely affect performance of said devices unless shielded properly. Another issue of radial movement of the linear pointer in a perpendicular plane to an ultrasound transducer is that it produces oblique presentations to a portion of a linear axis of an ultrasound transducer array. As the majority of ultrasonographic images are generated by volume averaging methods, the oblique placement of the linear pointer in relation to a linear axis of the ultrasound transducer array may limit production of an accurate linear shadow line in a visualized ultrasonographic field. This could produce ultrasonographic artifacts such as comet tail artifacts or side lobe artifacts, which may affect quality of imaging of the linear shadow line adversely.

### SUMMARY OF THE INVENTION

The present invention provides a static pointing device which does not require an electromagnetic pointing mechanism. The static pointing device comprises a stationary linear pointer fixedly embedded in a plate which is ultrasound-transmissible. The plate is placed perpendicularly to and in front of an ultrasound transducer and is to contact a skin overlying a tissue object. The stationary linear pointer runs in parallel with a linear axis of an ultrasound transducer array and is configured to block a portion of ultrasound waves emanating from an ultrasound transducer toward the tissue object.

In one embodiment, the static pointing device is configured in a flat rectangular plate and provided as a solid gel couplant in which the stationary linear pointer is embedded. In other embodiment, the static pointing device is configured in a curved plate to accommodate a curved ultrasound transducer head and similarly provided as a solid gel couplant. The gel couplant is releasably housed in an enclosure of an ultrasound positioning guide apparatus and is located proximally to an ultrasound transducer head. The gel couplant is configured to facilitate transmission of ultrasound waves from the ultrasound transducer to a tissue and is non-reusable. A transverse axis of the gel couplant is aligned with a longitudinal axis of the stationary linear pointer and with a linear axis of an ultrasound transducer array.

In one embodiment, the plate is made of one or a plurality of polymers, provided as one layer of sheet or stacked-up layers of sheet, which can be tightly attached to a face of the ultrasound transducer. A stationary linear pointer is embedded in the single-layered polymeric plate or is embedded in one layer of polymeric sheet which is sandwiched between at least two layers of sheet made of the same polymer(s) as for the stationary linear pointer sheet. The plate is configured to accommodate a shape of the face of the ultrasound transducer to cover an entire contact surface of said face. The plate can be made either permanently attached to the face of the ultrasound transducer or non-reusable.

In one embodiment, the stationary linear pointer is provided as a thin straight longitudinal bar which runs in parallel with both the transverse axis of the static pointing device and linear axis of the ultrasound transducer array and is embedded in the static pointing device. A transverse cross-section of the stationary linear pointer is configured in a box shape, a V shape or a semi-circular shape. An apex of the V shaped cross-section of the stationary linear pointer points to a skin and both open ends in cross section point to a face of the ultrasound transducer. Similarly, a convex portion of the semi-circular cross section points to the skin and both open ends in cross section point to the face of the ultrasound transducer. These configurations are to optimize blockade of ultrasound transmission emanating from the ultrasound transducer.

In one embodiment, the static pointing device is configured to be separate from and to be reversibly combined with an ultrasound pointing apparatus which comprises an invasive tubular device positioning guide, a position alignment assembly and a power and electronic control assembly. The invasive tubular device positioning guide is configured to provide a conduit through which an invasive tubular device passes, to be releasably attachable to the position alignment assembly and to be pivotably rotated for angulation of said invasive tubular device positioning guide toward a tissue object by said position alignment assembly. The position alignment assembly comprises a worm drive assembly and a rotary position sensor such as potentiometer, optical encoder or magnetic encoder connected to the worm drive assembly to measure rotations of said worm drive assembly, and an electric motor assembly to rotate the worm drive assembly if the apparatus is configured for a powered operation. The worm drive assembly is releasably connected to the invasive tubular device positioning guide and is configured to rotate the invasive tubular device about a pivot of said invasive tubular device. The power and electronic control assembly provides the apparatus with electricity and coordinates pivoting rotation of the invasive tubular device to be angulated toward the tissue object.

In one embodiment, an ultrasound transducer is enclosed in an enclosure which is configured to align longitudinal and transverse axes of the transducer in parallel with longitudinal and transverse axes of said transducer housing enclosure, respectively. The transverse axis of the transducer is used as a reference axis for the pivot of the invasive tubular device positioning guide to calibrate pivoted angulation of the invasive tubular device positioning guide and the longitudinal axis of the transducer is used as a reference axis for the invasive tubular device positioning guide to align a longitudinal axis of said invasive tubular device positioning guide with said longitudinal axis of the transducer.

In one embodiment, the rotary position sensor is provided as multi-turn sensor, which measures a degree of rotation of a worm shaft of the worm drive assembly which pivotably rotates the invasive tubular device about the pivot of the invasive tubular device positioning guide. The position sensor is electronically connected to the power and electronic control assembly which translates the degree of rotation of the worm shaft registered by said position sensor to a calculated distance between a contact portion of the face of the ultrasound transducer with a skin and a tissue object where a longitudinal line between said face of the transducer and said tissue object crosses a longitudinal axis of the invasive tubular device at an angle. The stationary linear pointer provides the ultrasound positioning apparatus with a visible linear shadow line in a visualized ultrasonographic view, which produces the longitudinal line between the face of the ultrasound transducer and the point in the tissue. When the linear shadow line crosses a tissue object, a distance from the face of the ultrasound transducer to the tissue object along the linear shadow line can accurately be measured by standard ultrasonographic machines. If the calculated distance based on the degree of the rotation of the worm drive assembly pivoting the invasive tubular device equals the measured distance between the face of the transducer and the tissue object, a calculated angle between the longitudinal axis of the invasive tubular device and the transverse axis of the ultrasound transducer should allow the longitudinal axis of the invasive tubular device to cross the linear shadow line at the tissue object. In this way, the invasive tubular device is predicted to be inserted to the tissue object before the invasive tubular device is introduced into the tissue. Once the invasive tubular device is visualized in the ultrasonographic view on its way toward the tissue object, a final location of the invasive tubular device can be confirmed in the visualized ultrasonographic view.

In one embodiment, the power and electronic control assembly is provided with a segment digital display which can be visualized. An integrated circuit board is located under and electronically connected to the segment digital display. A power source is provided to supply electricity to and interconnect the integrated circuit board, the segment digital display and the position sensor of the worm drive assembly, and the electric motor assembly if the apparatus is configured for a powered operation. A distance between the face of the transducer and the tissue object is measured by an operator and a digitized numerical information of the measured distance is put into the integrated circuit board which displays the numerical data in one part of the segment digital display. The operator then rotates the worm drive assembly to pivotably angulate the invasive tubular device positioning guide, which is monitored as a varying calculated distance between the face of the ultrasound transducer and a point in the tissue where the longitudinal axis of the invasive tubular device crosses the linear shadow line originating from the stationary linear pointer. The calculated distance is displayed in the segment digital display below the displayed measured distance. When the calculated distance matches the measured distance, the longitudinal axis of the invasive tubular device is to cross the linear shadow line at the tissue object. The calculated distance is variable over a predetermined distance for a particular ultrasound positioning apparatus based on the tissue penetration depth of an ultrasound transducer.

In one embodiment, a distance (a) from a face of an ultrasound transducer to a center of a tissue object is calculated by a substantially tangential placement of the face of the transducer to a skin overlying the tissue object. A horizontal distance from a pivot of an invasive tubular device positioning guide to a stationary linear pointer measures as (b). Using a simple trigonometry, a distance (h) of the invasive tubular device from the pivot of the invasive tubular device positioning guide to the center of the object equals a square root of (a²+b²). A sine of an angle (α) of the invasive tubular device relative to a transverse axis of the ultrasound transducer is calculated as a ratio of (a) to (h). As the (b) is a fixed value, the only variable affecting the angle (α) of the invasive tubular device is the distance (a) which can be measured prior to inserting the invasive tubular device. In another embodiment, a degree of pivotable angulation of the invasive tubular device by the worm is dependent on a ratio of a unit change (Δ) in sine of an angle (α) of the invasive tubular device to one revolution of the worm which is monitored by the position sensor.

### BRIEF DESCRIPTION OF THE DRAWINGS

FIGS. 1A, 1B, 1C and 1D show a schematic illustration of an example of individual components of an apparatus with which a static pointing device is releasably combined. FIG. 1A represents an example of a pivotable invasive tubular device positioning guide; FIG. 1B represents an ultrasound transducer head; FIG. 1C represents a position alignment assembly; FIG. 1D represents a static pointing device.

FIGS. 2A and 2B show a schematic example of the position alignment assembly and the static pointing device for a flat ultrasound transducer head: FIG. 2A shows individual compartments inside the position alignment assembly with a transducer housing removed for illustration; FIG. 2B shows a flat rectangular static pointing device with a stationary linear pointer embedded inside.

FIGS. 3A and 3B show a schematic example of the position alignment assembly and a curved static pointing device for a curved ultrasound transducer head: FIG. 3A shows an in-situ placement of the curved static pointing device with a transducer housing removed for illustration; FIG. 3B shows the curved static pointing device with a stationary linear pointer embedded inside.

FIG. 4 shows a schematic example of a static pointing device fixedly attached to a face of an ultrasound transducer.

FIGS. 5A, 5B and 5C illustrate a schematic example of a method of coordination of a pivoted rotation of the invasive tubular device positioning guide with a distance over a range between a face of the ultrasound transducer and a tissue object along a linear shadow line produced by the stationary linear pointer. An upper box drawing of each FIGS. 5A, 5B and SC represents a schematic segment digital display showing numerical data of distance. A mid drawing of each FIGS. 5A, 5B and 5C shows a schematic profile view of the apparatus. A lower drawing of each FIGS. 5A, 5B and 5C depicts a schematic ultrasonographic two-dimensional view seen in a monitor of an ultrasonographic machine.

FIGS. 6A, 6B and 6C show a schematic illustration of an example of individual components of a powered stereotactic positioning guide apparatus, including a static pointing device; FIG. 6A shows an invasive tubular device positioning guide having a tubular guide; FIG. 6B shows a slidably insertable static pointing device; FIG. 6C shows a schematic external view of the powered stereotactic positioning apparatus.

FIGS. 7A and 7B depict a schematic illustration of internal components housed in the powered stereotactic positioning guide apparatus and the static pointing device; FIG. 7A shows a gearbox assembly connected to an output shaft gear assembly; FIG. 7B shows a static pointing device located in front of an ultrasound transducer, said ultrasound transducer attached to a handle and an electronic control assembly located distally to said ultrasound transducer.

FIGS. 8A, 8B and 8C illustrate a schematic example of a method of coordination of a pivoted rotation of the invasive tubular device positioning guide attached to the powered stereotactic positioning guide apparatus with a distance over a range between a face of an ultrasound transducer and a tissue object along a linear shadow line produced by a stationary linear pointer. An upper box drawing of each FIGS. 8A, 8B and 8C represents a schematic segment digital display showing numerical data of distance. A mid drawing of each FIGS. 8A, 8B and 8C shows a schematic profile view of the apparatus. A lower drawing of each FIGS. 8A, 8B and 8C depict a schematic ultrasonographic two-dimensional view seen in a monitor of an ultrasonographic machine.

### DETAILED DESCRIPTION OF THE DRAWINGS

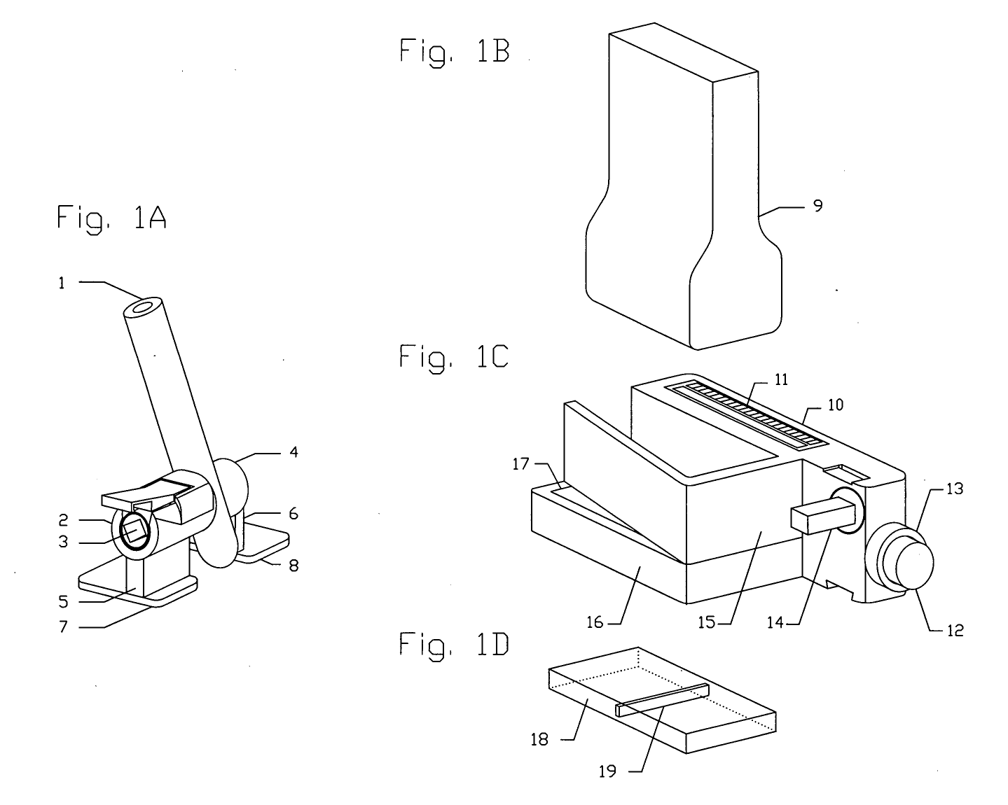

As described below, the present invention provides a static pointing device assisting an ultrasound positioning guide for directing an invasive tubular device toward a tissue object. It is to be understood that the descriptions are solely for the purposes of illustrating the present invention, and should not be understood in any way as restrictive or limited. Embodiments of the present invention are preferably depicted with reference to FIGS. 1 to 8, however, such reference is not intended to limit the present invention in any manner. The drawings do not represent actual dimension of devices, but illustrate the principles of the present invention.

FIGS. 1A, 1B, 1C and 1D show a schematic example of individual components of an ultrasound positioning apparatus and a static pointing device of the present invention. FIG. 1A represents an example of a pivotable invasive tubular device positioning guide which comprises a tubular guide 1, an outer rotation cylinder 2, a slot 3 for an output shaft of a worm drive assembly, a stabilization cylinder 4, a pair of attachment bars 5 and 6 and a pair of bottom plates 7 and 8. In FIG. 1B, an ultrasound transducer head 9 is shown which can be releasably held by an ultrasound transducer holder 15 shown in FIG. 1C. FIG. 1C shows a position alignment assembly which comprises a positioning controller assembly 10, a segment digital display 11, a rotatable knob 12 to rotate the worm drive assembly inside the positioning controller assembly 10, a second outer rotatable knob 13 to provide a power and electronic control assembly with numerical input such as measured distance, the output shaft 14 of the worm drive assembly, the ultrasound transducer holder 15, an enclosure 16 showing an inner wall 17 to accommodate a face portion of the ultrasound transducer and a static pointing device. FIG. 1D shows an example of a static pointing device 18 which has a stationary linear pointer 19 embedded in said static pointing device along a transverse axis of said pointing device. The static pointing device 18 is placed perpendicularly to the face of the ultrasound transducer 9 inside the enclosure 16.

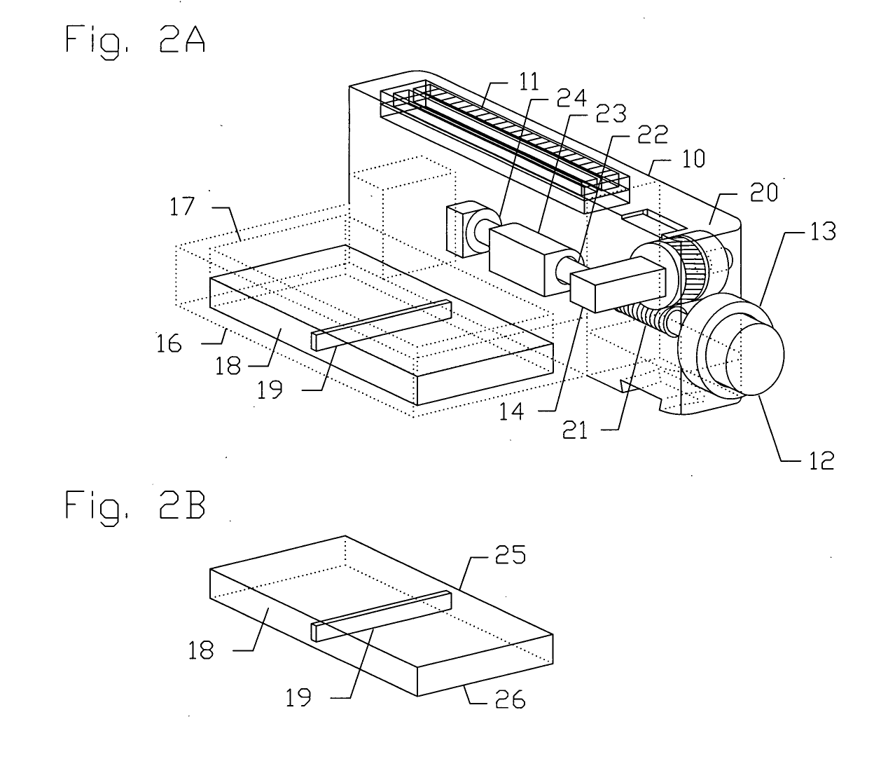

FIGS. 2A and 2B show a schematic example of the position alignment assembly and the static pointing device for a flat ultrasound transducer head. FIG. 2A shows individual compartments inside the position alignment assembly with a transducer housing removed for illustration. In a proximal portion 20 of the position alignment assembly 10, there is provided the worm drive assembly with a worm 21 coaxially connected to a multi-turn rotary position sensor 24 via a connecting shaft 22. The connecting shaft 22 is housed by a shaft anchoring portion 23 attached to an inner wall of the position alignment assembly. The rotatable knob 12 rotates the worm 21 which in turn rotates a worm gear mated with the worm 21. Rotations of the worm are sensed by the multi-turn rotary position sensor 24 and the output shaft 14 connected to the worm gear transmits rotations of the worm gear to the rotation cylinder 2 shown in FIG. 1A. The static pointing device 18 is placed in the enclosure 16, and the stationary linear pointer 19 is aligned in parallel with a longitudinal axis of the output shaft 14. Shown in FIG. 2B, the flat rectangular static pointing device 18 has an upper surface 25 which comes in contact with the face of the ultrasound transducer and a lower surface 26 which comes in contact with a skin overlying a tissue object.

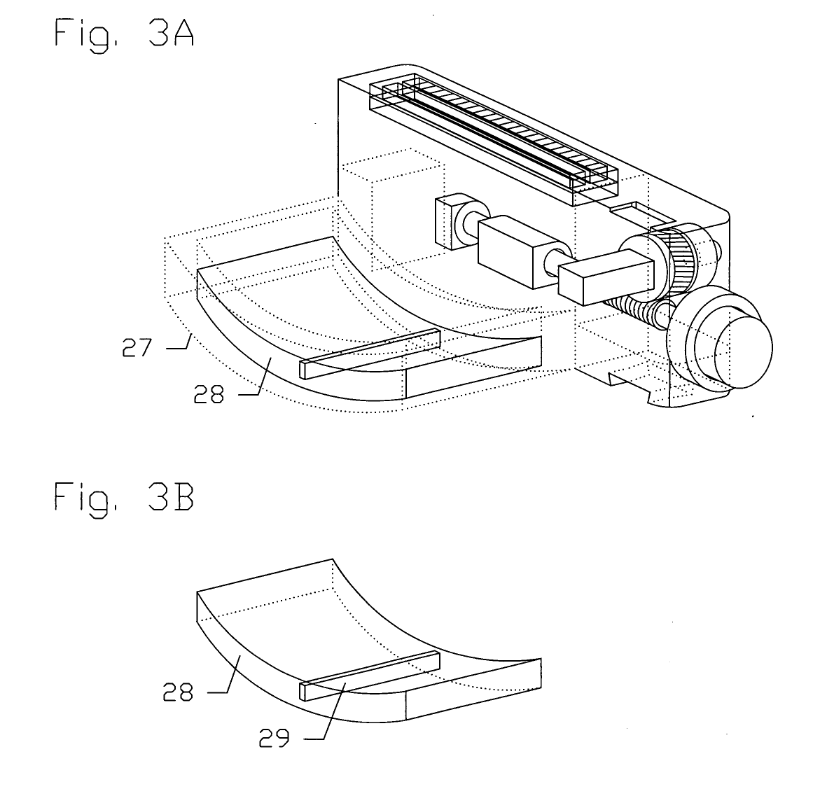

FIGS. 3A and 3B show a schematic example of the position alignment assembly and a curved static pointing device for a curved ultrasound transducer head. FIG. 3A shows an in-situ placement of the curved static pointing device 28 in a curved enclosure 27 with a transducer housing removed for illustration. FIG. 3B shows the curved static pointing device 28 with a stationary linear pointer 29 embedded inside in said static pointing device.

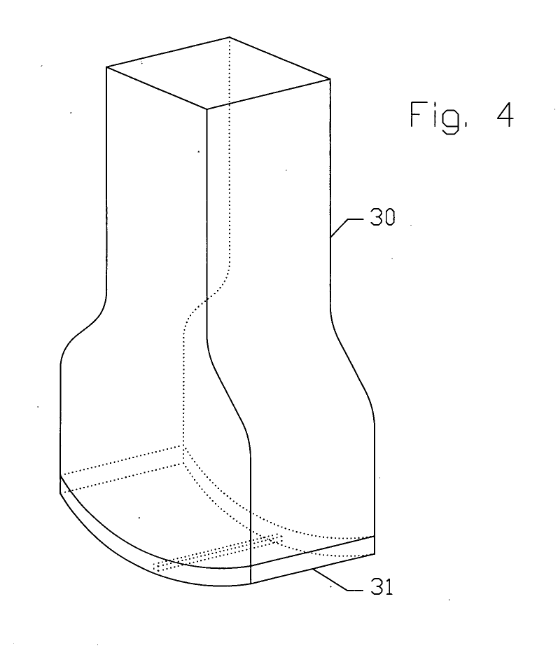

FIG. 4 shows a schematic example of a static pointing device 31 in a curved plate configuration fixedly attached to a curved face of an ultrasound transducer 30. In this example, the curved plate 31 made of one or a plurality of polymers is provided as one layer of sheet which is tightly attached to the face of the ultrasound transducer 30. A stationary linear pointer is embedded in the single-layered polymeric plate, with its longitudinal axis in parallel with a linear axis of a transducer array. The plate covers an entire contact surface of said face.

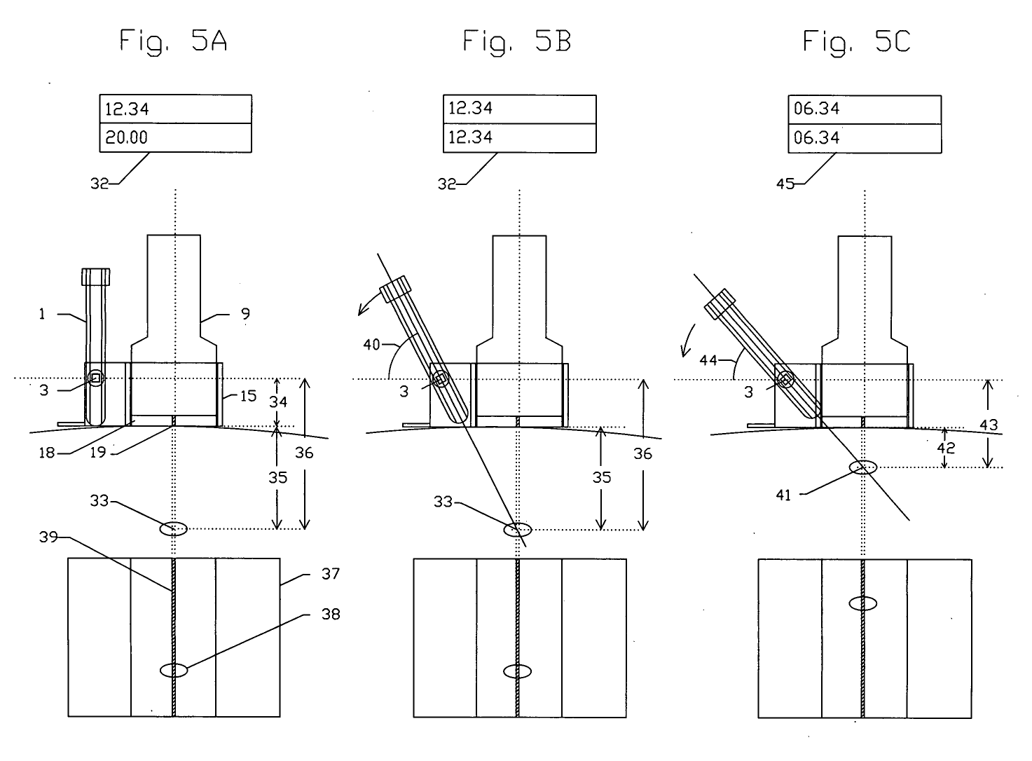

FIGS. 5A, 5B and 5C illustrate a schematic example of a method of coordination of a pivoted rotation of the invasive tubular device positioning guide with a distance over a range between a face of the ultrasound transducer and a tissue object along a linear shadow line produced by a stationary linear pointer. Upper box drawings 32 and 45 in FIGS. 5A, 5B and 5C represent a schematic segment digital display showing numerical data of distance. Mid drawings in FIGS. 5A, 5B and 5C show a schematic profile view of the apparatus with an ultrasound transducer 9 held in place by a holder 15 placed atop a skin overlying tissue objects 33 and 41. Lower drawings in FIGS. 5A, 5B and 5C depict a schematic ultrasonographic two-dimensional view 37 seen in a monitor of an ultrasonographic machine. As illustrated in FIG. 5A, once the apparatus is placed on the skin above the tissue object 33, a stationary linear pointer 19 of a static pointing device 18 generates a linear blank shadow line 39 in the two-dimensional view 37 going through an ultrasonographic image 38 of the tissue object 33 by blocking off a portion of ultrasound waves. A longitudinal axis of a tubular guide 1 of an invasive tubular device is aligned in parallel with the linear shadow line 39 by default. An upper row of a digital display of the upper box drawing 32 represents a measured distance 35 put in by an operator. In this particular example, it shows a distance of 12.34 centimeters. A pivotable distance 36 for calculation of a calculated distance comes from a sum of the measured distance 35 and a fixed value of a vertical distance 34 between the contact portion of the transducer and a pivot 3 of the invasive tubular device positioning guide. A lower row of the upper box drawing 32 shows a default distance set by a farthest tissue penetration depth by a particular ultrasound transducer. In this example, it is set at 20.00 centimeters.

In FIG. 5B, the tubular guide 1 is pivotably rotated about the pivot 3 by a worm drive assembly shown in FIG. 2A, which is monitored numerically as calculated distance in the lower row of the upper drawing 32 of the segment digital display. The calculated distance is a distance along the linear shadow line between the contact portion of the transducer with the skin and a point in the tissue where the longitudinal axis of the tubular guide 1 crosses said linear shadow line. The calculated distance is configured to decrease from the farthest distance to the measured distance. In this example, the calculated distance decreases from the 20.00 centimeters of the farthest distance measurable by the ultrasound transducer 9 to the 12.34 centimeters of the measured distance to indicate an angulation 40 of the longitudinal axis to the tissue object 33. The pivoted angle 40 when the calculated distance equals the measured distance is calculated using a numerical information of the distance but is not actually measured.

FIG. 5C shows a schematic example of a tissue object 41 located closer to the skin than the object 33 of FIGS. 5A and 5B. A measured distance 42 is shorter at 6.34 centimeters, displayed in an upper row of the upper box drawing 45. A pivotable distance 43 for calculation of a calculated distance is a sum of the measured distance 42 and the same fixed distance as 34 of FIG. 5A. Similar to FIG. 5B, the calculated distance decreases from the 20.00 centimeters of the farthest distance measurable by the ultrasound transducer 9 to the 6.34 centimeters of the measured distance to indicate an angulation 44 of the longitudinal axis to the tissue object 41. Similar to FIG. 5B, the pivoted angle 44 when the calculated distance equals the measured distance is calculated using a numerical information of the distance but is not actually measured.

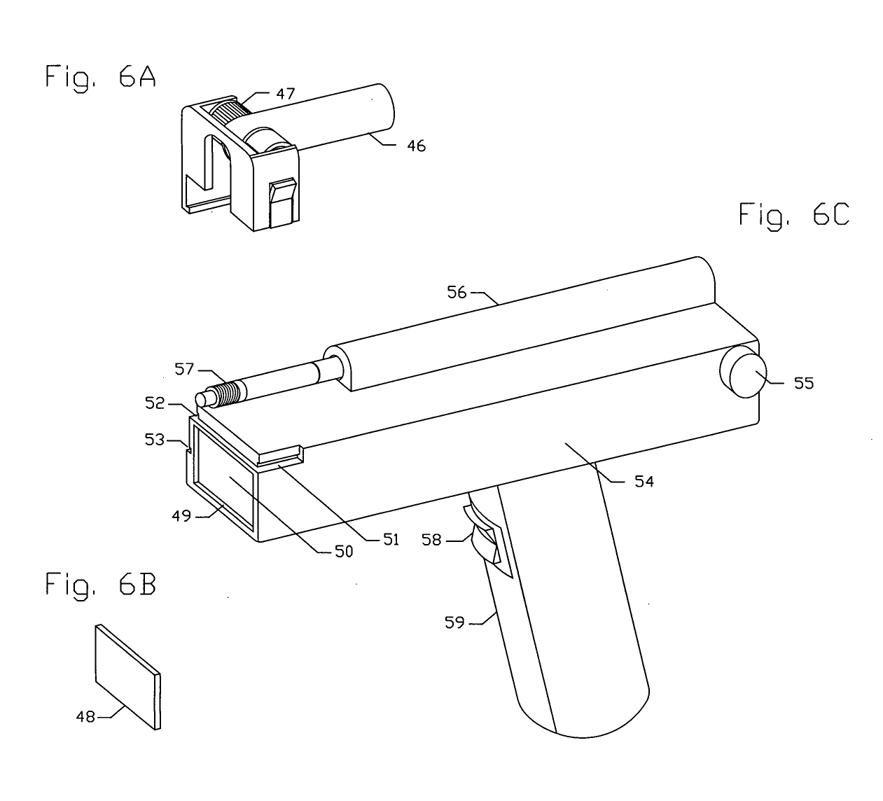

FIGS. 6A, 6B and 6C show a schematic illustration of an example of individual components of a powered stereotactic positioning guide apparatus, including a static pointing device. FIG. 6A shows an invasive tubular device positioning guide having a tubular guide 46 through which an invasive tubular device passes and a worm gear 47 which meshes with a worm 57 shown in FIG. 6C of a powered stereotactic positioning apparatus. FIG. 6B shows a static pointing device 48 which is slidably insertable to an enclosure 49 located at a proximal end of the powered stereotactic positioning apparatus shown in FIG. 6C. The static pointing device 48 is placed perpendicularly to and in direct contact with the face 50 of the ultrasound transducer. FIG. 6C shows a schematic external view of the powered stereotactic positioning apparatus, having a face portion of an ultrasound transducer 50, a group of notches 51, 52 and 53 for anchoring the invasive tubular device positioning guide of FIG. 6A, a main body 54 to house internal components, a rotatable knob 55 to put in numerical information to a power and electronic control assembly, an outer semi-circular tube 56 to house an output shaft delivering rotation torque to the worm 57, the worm 57 coaxially connected to the output shaft to rotate the worm gear 47 which in turn pivotably rotates the tubular guide 46, an electric switch 58 to power and control rotation of the worm 57 and a handle 59 which houses electric cables connecting the ultrasound transducer to a main ultrasound machine.

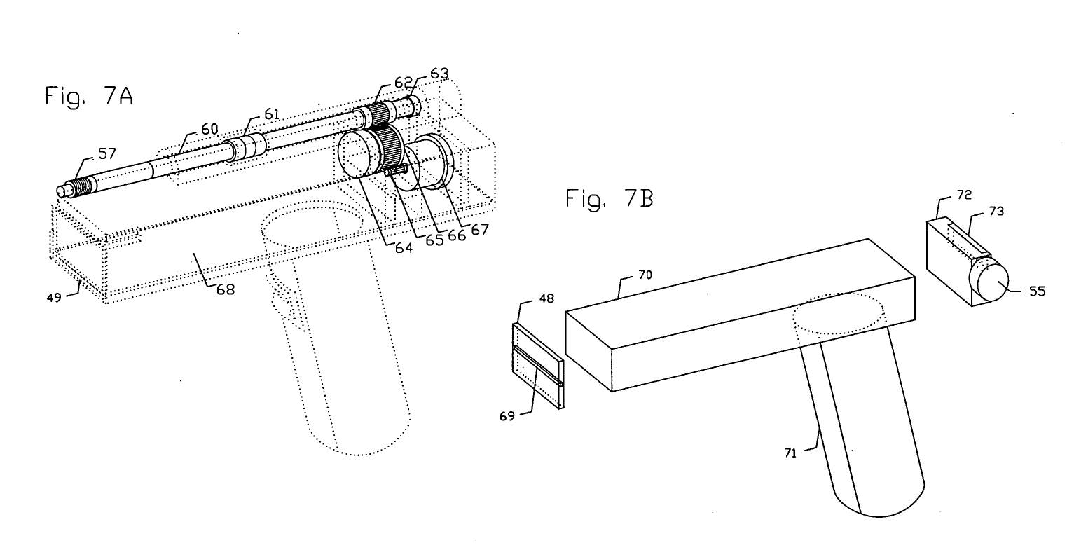

FIGS. 7A and 7B depict a schematic illustration of internal components housed in the powered stereotactic positioning guide apparatus and the static pointing device. FIG. 7A shows the worm 57 connected to the output shaft 60 which is coaxially connected to a spur gear 62. Rotation of the output shaft 60 is assisted by a rolling-element bearing arrangement 61 which is configured to reduce friction between the output shaft enclosure and the output shaft. A central tubular cup 63 is provided inside the output shaft enclosure to accommodate a distal end of the output shaft for axial rotation. The spur gear 62 of the output shaft meshes with a second spur gear 66 which is configured to be rotated by a rotor 65 mated in parallel with said spur gear 66. The rotor 65 protrudes from an electric servomotor 67 which is electronically connected with a multi-turn rotary position sensor 64. The multi-turn rotary position sensor 64 is coaxially connected with the second spur gear 66 and monitors rotations of said spur gear 66. In front of the motor assembly, there is provided a rectangular tubular space 68 which houses an ultrasound transducer 70 shown in FIG. 7B. The static pointing device 48 shown in FIG. 7B is slidably and reversibly insertable to the enclosure 49, and is in direct contact with the face of the ultrasound transducer 70. A stationary linear pointer 69 is embedded in the static pointing device, with its longitudinal axis in parallel with a linear axis of an ultrasound transducer array. The ultrasound transducer 70 is connected to a handle portion 71 which carries cables to connect the ultrasound transducer to the main ultrasound machine. Behind the motor assembly, there is provided an electronic control assembly 72 which comprises an integrated circuit board, the rotatable knob 55 and a segment digital display 73. The segment digital display 73 is configured to be visible outside the powered stereotactic positioning guide apparatus.

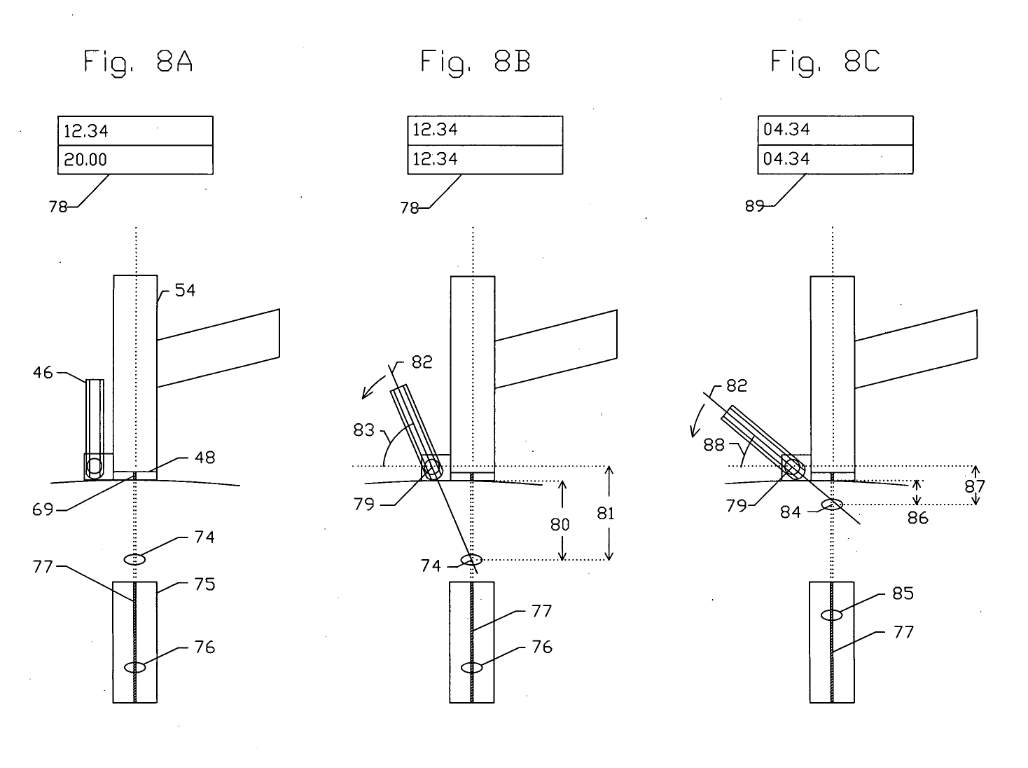

FIGS. 8A, 8B and 8C illustrate a schematic example of a method of coordination of a pivoted rotation of the tubular guide 46 the invasive tubular device positioning guide attached to the powered stereotactic positioning guide apparatus 54. Upper box drawings 78 and 89 in FIGS. 8A, 8B and 8C represent a schematic segment digital display showing numerical data of distance. Mid drawings in FIGS. 8A, 8B and 8C show a schematic profile view of the apparatus 54 housing an ultrasound transducer placed atop a skin overlying tissue objects 74 and 84. Lower drawings in FIGS. 8A, 8B and 8C depict a schematic ultrasonographic two-dimensional view 75 seen in a monitor of an ultrasonographic machine. As illustrated in FIG. 8A, once the apparatus is placed on the skin above the tissue object 74, a stationary linear pointer 69 of a static pointing device 48 generates a linear blank shadow line 77 in the two-dimensional view 75 going through an ultrasonographic image 76 of the tissue object 74 by blocking off a portion of ultrasound waves. A longitudinal axis of the tubular guide 46 of an invasive tubular device is aligned in parallel with the linear shadow line 77 by default.

In FIG. 8B, an upper row of a digital display of the upper box drawing 78 represents a measured distance 80 put in by an operator. In this particular example, it shows a distance of 12.34 centimeters. The motor assembly shown in FIG. 7A pivotably rotates the tubular guide 46, which is monitored numerically as calculated distance in the lower row of the upper drawing 78 of the segment digital display. Before pivotable rotation of the tubular guide, a default distance set by a farthest tissue penetration depth by a particular ultrasound transducer is shown in a lower row of the upper box drawing 78 as illustrated in FIG. 8A. In this particular example, it shows a distance of 20.00 centimeters. When the tubular guide is pivotably rotated in FIG. 8B, the calculated distance decreases from the farthest distance of 20.00 centimeters to the measured distance of 12.34 centimeters as a longitudinal axis 82 of the tubular guide 46 approaches the tissue object 74 along the linear shadow line 77. In FIG. 8B, a distance 81 represents the pivotable distance for calculation of a calculated distance, which is a sum of the measured distance 80 and a fixed value of a vertical distance between the contact portion of the transducer and a pivot 79 of the invasive tubular device positioning guide. A pivoted angle 83 when the calculated distance equals the measured distance is calculated using a numerical information of the distance but is not actually measured.

FIG. 8C shows a schematic example of a tissue object 84 located closer to the skin than the object 74 of FIGS. 8A and 8B. A measured distance 86 between the contact portion of the ultrasound transducer and an ultrasonographic tissue image 85 is shorter at 4.34 centimeters, displayed in an upper row of the upper box drawing 89. A pivotable distance 87 for calculation of a calculated distance is a sum of the measured distance 86 and the fixed value of a vertical distance between the contact portion of the transducer and the pivot 79 of the invasive tubular device positioning guide. Similar to FIG. 8B, the calculated distance shown in the lower row of the upper box drawing 89 decreases from the 20.00 centimeters of the farthest distance measurable by the ultrasound transducer to the 4.34 centimeters of the measured distance as the longitudinal axis 82 of the tubular guide approaches the tissue object 84 along the linear shadow line 77. A pivoted angle 88 when the calculated distance equals the measured distance is calculated using a numerical information of the distance but is not actually measured.

It is to be understood that the aforementioned description of the apparatus and methods is simple illustrative embodiments of the principles of the present invention. Various modifications and variations of the description of the present invention are expected to occur to those skilled in the art without departing from the spirit and scope of the present invention. Therefore the present invention is to be defined not by the aforementioned description but instead by the spirit and scope of the following claims.

What is claimed is:

1. A static pointing device, comprising:
a plate, and a stationary linear pointer;
the plate, having a relatively broad surface in relation to thickness, which is made of one or a plurality of substantially ultrasound-transmissible polymers, which is configured to cover a face of an ultrasound transducer, which is placed perpendicularly to a longitudinal axis of the ultrasound transducer enclosed in an ultrasound positioning apparatus for an invasive tubular device and which is placed in between of and in contact with the face of the ultrasound transducer and a tissue of a living body; and
the stationary linear pointer, provided as a thin longitudinal bar, which has one or a plurality of configurations on a transverse cross section of said bar, which is aligned in parallel with a transverse axis of the plate and with a linear axis of an ultrasound transducer array, which is fixedly embedded in the plate and which is configured to produce a linear shadow line in a visualized ultrasonographic field by blocking ultrasound waves which are to pass through said stationary linear pointer from the ultrasound transducer toward the tissue.

2. The static pointing device according to claim 1, wherein the at least one plate is a solid ultrasound gel couplant which is separate from and in direct contact with an ultrasound transducer.

3. The static pointing device according to claim 1, wherein the at least one plate is made permanently attached to the face of the ultrasound transducer.

4. A method for the static pointing device according to claim 1, wherein the linear shadow line between the at least one stationary linear pointer and a tissue object produced by ultrasound waves emanating from the ultrasound transducer in an visualized ultrasonographic field serves as a reference axis on which the ultrasound positioning apparatus relies for calculation of an insertion path for an invasive tubular device attached to said ultrasound positioning apparatus toward the tissue object prior to tissue penetration by said invasive tubular device.
